(19) United States
(12) Patent Application Publication
(10) Pub. No.: US 2010/0028281 A1
(43) Pub. Date: Feb. 4, 2010

(54) Hair care composition

(76) Inventors: Teresa Robinson, Bebington (GB); Cheryl Anne Taylor, Wirral (GB)

Correspondence Address: UNILEVER PATENT GROUP, 800 SYLVAN AVENUE, AG West S. Wing, ENGLEWOOD CLIFFS, NJ, 07632-3100, US

(21) Appl. No.: 12/309,308

(22) PCT Filed: Jun. 29, 2007
(86) PCT No.: PCT/EP2007/056593

§ 371 Date: Sep. 1, 2009

(30) Foreign Application Priority Data
Jul. 14, 2006 (EP) 06253701.4

(51) Int. Cl. A61K 8/18; A61Q 5/04

(52) U.S. Cl. 424/70.2

(57) ABSTRACT

A post-treatment composition comprising at least one basic amino acid, in which said post-treatment composition is applied to hair after applying a relaxing composition.

### FIELD OF THE INVENTION

The present invention is directed to a hair relaxing composition and a method of relaxing hair.

### BACKGROUND OF THE INVENTION

Hair relaxers are compositions used to relax or straighten curly or kinky hair. Most hair relaxers straighten hair by disrupting disulfide bonds of the hair fibres with an alkaline agent or reducing agent. The chemical disruption of disulfide bonds is usually combined with mechanical straightening of the hair (eg by combing). The straightening process is generally terminated by rinsing and/or the application of a neutralizing composition.

A source of hydroxide ions is usually the preferred alkaline agent used to straighten hair. The term “lanthionizing” is used when referring to hair relaxed or straightened by hydroxide ions, as the straightening reaction sequence with hydroxide ions results in lanthionine residue formation.

Most frequently, commercial relaxing compositions are in the form of gels or emulsions and contain varying proportions of strong water-soluble bases, such as sodium hydroxide (NaOH). Also used are slightly-soluble metal hydroxides, such as calcium hydroxide (Ca(OH)₂), which can be converted in situ to soluble bases, such as guanidine hydroxide.

A key problem with hair relaxers is that they leave the hair treated therewith feeling rough and unconditioned.

WO 02/085317 discloses compositions and methods for lathionizing keratin fibres using at least one organic nucleophile and at least one hydroxide ion generator. WO 02/085317 discloses that the organic nucleophile is especially effective when used in a pre-treatment composition.

The present invention relates to hair relaxing systems which leave the hair feeling smooth and soft feel, and easily combed.

### DESCRIPTION OF THE INVENTION

In one aspect the present invention relates to a post-treatment composition comprising at least one basic amino acid, in which said post-treatment composition is applied to hair after applying a relaxing composition.

In a second aspect the invention relates to a method for relaxing hair comprising the following steps:
i) applying to the hair for a sufficient period of time to lanthionize the hair a relaxer composition;
ii) terminating the lanthionization process;
iii) applying to the lanthionized hair a composition comprising a basic amino acid.

A further aspect of the invention is the use of a basic amino acid in a post-treatment composition for improving the soft feel, the smooth appearance or the ease of combing of hair.

### DETAILED DESCRIPTION OF THE INVENTION

As disclosed above the present invention relates to post-treatment composition for application after application of a relaxing composition. In the context of the present invention “relaxing composition” means a composition comprising at least one hydroxide ion generator in an amount sufficient to effect lanthionization of keratin fibres. The term post-treatment refers to a treatment applied to the hair after the lathionization process, and preferably after any subsequent termination process.

Basic amino acid refers to a natural and synthetic basic amino acids, their isomeric and racemic forms, and derivatives thereof. It is preferred if the basic amino acid is chosen from, lysine, arginine, ornithine or histidine, which may be in a non-ionic form (such as lysine) and/or in the form of derivatives thereof e.g., an ammonium form (such as lysine hydrochloride) and/or a carboxylate form. Particularly preferred are lysine, histidine, ornithine or arginine in their ionic form, lysine is the most preferred.

The basic amino acid is preferably present in the post-treatment composition in an amount ranging from 0.5% to 10.0% by weight relative to the total weight of the post-treatment composition, more preferably at a level greater than 3 wt %, most preferably at a level from 3.5 to 7.5 wt %.

The hydroxide ion generator may be chosen from those compositions that produce hydroxide ions appropriate for the lanthionization of hair. As used herein, “hydroxide ion generator” refers to both compounds and compositions that generate hydroxide ions, and compounds and compositions that comprise hydroxide ions. Hydroxide ion generators may, for example, be chosen from traditional “Iye” and “no lye” hair relaxer compositions and other soluble or slightly soluble hydroxide ion sources. Preferred hydroxide ion generators are strong water-soluble bases, particularly is sodium hydroxide.

Compositions according to the inventive may comprise at least one further constituent such as alcohol, water and propellants; preservatives; perfumes; anionic, cationic, amphoteric, nonionic, and zwitterionic surfactants; hair conditioning agents such as silicone fluids, fatty esters, fatty alcohol, fatty chain hydrocarbons, emollients, lubricants, and penetrants such as lanolin compounds, protein hydrolysates, and other protein derivatives; anionic, cationic, amphoteric, nonionic, and zwitterionic polymers; colourants; bleaches; reducing agents; pH adjusting agents; sunscreens; styling polymers and thickening agents.

This post treatment composition may be in any form preferably in the form chosen from emulsions, solutions, suspensions, gels, creams, and pastes.

The compositions of the present invention may also be provided as a multi-component kit for straightening hair comprising at least two separate components. A first component of the kit contains comprises at least one composition for generating hydroxide ions to relax the hair. The second component comprises at least one composition comprising at least one basic amino acid.

The process of treating the hair comprises the steps of:

i) applying to the hair for a sufficient period of time to lanthionize the hair a relaxer composition;
ii) terminating the lanthionization process;
iii) applying to the lanthionized hair a composition comprising a basic amino acid.

A preferred method of terminating the lanthionization process is by rinsing the hair with water.

The invention will now be illustrated by the following non-limiting Examples.

### Examples

A series of switches were treated with lysine either prior to relaxing, during relaxing or after relaxing and were assessed for sensory attributes in direct comparison to switches that have been treated by just sodium hydroxide alone.

### Pre Treating

2.5 gm 6″ wide clip mulatto switches supplied from International Hair Importer was treated to a pre-treatment of 5% lysine solution. The switches were bathed in 150 mls solution of the 5% lysine for 15 minutes prior to being treated to a 3.5% aqueous solution of sodium hydroxide for 30 minutes. The switches were not rinsed when they were transferred from the lysine solution to the sodium hydroxide solution. After the switches had been immersed in the aqueous sodium hydroxide solution the switches were then removed and then rinsed by being held under tepid running water for 1 minute. The switches were then lathered with laboratory prepared neutralising shampoo for 30 seconds prior to being rinsed and then dried in an oven for 45 minutes at 50° C.

### Control Conditions;

2.5 gm 6″ wide clip mulatto switches supplied from International Hair Importer were bathed in 150 mls of 3.5% aqueous sodium hydroxide for 30 minutes at ambient conditions. After the switches had been immersed in the aqueous sodium hydroxide solution the switches were then removed and then rinsed by being held under tepid running water for 1 minute. The switches were then lathered with laboratory prepared neutralising shampoo for 30 seconds prior to being rinsed and then dried in an oven for 45 minutes at 50° C.

### Post Treating

2.5 gm 6″ wide clip mulatto switches supplied from International Hair Importer was bathed in 150 ml of 3.5% aqueous sodium hydroxide for 30 minutes at ambient conditions. Following that the switches were then removed and then rinsed by being held under tepid running water for 1 minute. These switches were then immediately bathed in a 150 ml 5% lysine solution for 15 minutes. These switches were then rinsed, lathered using laboratory prepared neutralising shampoo for 30 seconds prior to being rinsed and dried in the oven for 45 minutes at 50° C.

### In-Situ Relaxing

2.5 gm 6″ wide clip mulatto switches supplied from International Hair Importer were bathed in 150 mls of a solution containing 3.5% aqueous sodium hydroxide and 5% lysine for 30 minutes at ambient conditions. After the switches had been immersed in the solution the switches were then removed and then rinsed by being held under tepid running water for 1 minute. The switches were then lathered with laboratory prepared neutralising shampoo for 30 seconds and rinsed again. Switches were then dried in the oven for 45 minutes at 50° C.

All chemicals used were supplied by Sigma Aldrich Chemicals

All the switches were then assessed for the following sensory attributes; smoothness, ease of comb and softness using the paired comparison Bradley-Terry analyses. (3 product testing)

Test 1: The following were directly compared to the control set (treated with 3.5% Sodium hydroxide), pre treatment with lysine and Post Treated with Lysine. For the preference scores for test 1 see table 1

TABLE 1
the preference scores for the analysis of pre treatment
compared against control and post treated switches.

| | Smoothness | Ease of comb | Softness |
|---|---|---|---|
| Treatment | preference | preference | preference |
| Pre treatment | 37% | 30% | 32% |
| with lysine | | | |
| Post treatment | 63% | 70% | 68% |
| with lysine | | | |

For all attributes the control set was scored as the worse and was never selected.

Test 2. The following were directly compared to the control set (treated with 3.5% Sodium hydroxide), post treated with lysine and the in-situ combination of lysine and sodium hydroxide. For the preference see table 2

| | Smoothness | Ease of comb | Softness |
|---|---|---|---|
| Treatment | preference | preference | preference |
| Post treatment | 66% | 91% | 70% |
| with lysine | | | |
| In-situ treatment | 33% | 8% | 30% |
| with lysine and | | | |
| sodium hydroxide | | | |

For all attributes the control set was scored as the worse and was never selected.

The formulations below are examples according to the invention of products for use after the straightening procedure.

| | | | |
|---|---|---|---|
| Trade Name | Chemical Name | Supplier | Nominal % |

Oil Moisturiser Formulation

| | | | |
|---|---|---|---|
| Oil Phase | | | |
| Superla No 7 | White Mineral Oil | Amoco | 35.00 |
| White Beeswax | Beeswax | Koster Keunen | 4.00 |
| Perfecta | Petroleum Jelly - | Crompton | 2.00 |
| Petrolatum | White | | |
| Sorbitan | Sorbitan | Sigma/Aldrich | 0.50 |
| Sesquiloleate | Sesquiloleate | | |
| Corona 8 | Lanolin | Croda | 5.00 |
| Water Phase | | | |
| Borax | Borax | Sigma/Aldrich | 0.67 |
| Water | | | to 80% |
| Water Soluble | | | |
| Active | | | |
| Lysine | Lysine HCl 80% | Sigma/Aldrich | 5.00 |
| | active | | |
| Water | | | to 20% |

Intense Conditioner

| | | | |
|---|---|---|---|
| Oil Phase | | | |
| Laurex CS | Cetyl/stearyl | Huntsman | 4.80 |
| | Alcohol | | |
| Perfecta | Petroleum Jelly - | Crompton | 0.10 |
| Petrolatum | White | | |
| Superla No 7 | White Mineral Oil | Amoco | 0.25 |
| CrodalanLA | Cetyl Acetate and | Croda | 0.90 |
| | Stearyl Acetate | | |
| | and Oleyl Acetate | | |
| | and Acetylated | | |
| | Lanolin Alcohol | | |
| Water Phase | | | |
| Arquad 16-29 | Cetrimonium | Akzo Nobel | 1.30 |
| | Chloride 29% | | |
| | (CTAC) | | |
| Glycerol | Glycerol | Sigma/Aldrich | 0.50 |
| Natrosol | Hydroxyethyl- | Hercules | 0.30 |
| 250HHR | celluslose | | |
| Water | | | to 50% |
| Quench water | | | 30.00 |
| Water Soluble | | | |
| Active/Additves | | | |
| to add when cool | | | |
| DC2-1785 | Silicone Emulsion | Dow Corning | 1.50 |
| Lysine | Lysine HCl | Sigma/Aldrich | 5.00 |
| | (80% Lysine) | | |
| Water | | | to 20% |
| Hot Oil Formula- | | | |
| tion + 5% Lysine | | | |
| HIGH | | | |
| Natrosol | Hydroxycellulose | Hercules | 0.70 |
| EDTA | Disodium | BASF | 0.10 |
| Glydant | DMDM Hydantoin | Lonza UL LTD | 0.10 |
| Lysine | Lysine HCl | Sigma/Aldrich | 5.00 |
| Water | | | to 100 |
| Citric | Citric Acid/NaOH | Sigma/Aldrich | to pH 4.5-5.0 |
| Acid/NaOH | | | |

1. A method for relaxing hair comprising the following steps:
i) applying to the hair for a sufficient period of time to lanthionize the hair a relaxer composition;
ii) terminating the lanthionization process;
iii) applying to the lanthionized hair a post treatment composition comprising 0.5 to 10 wt % of the total composition of a basic amino acid.

2. A method according to claim 1 in which the total level of basic amino acid is from 3.5 to 7.5 wt % of the total post-treatment composition.

3. A method according to claim 1 in which the basic amino acids are chosen from the group consisting of lysine, arginine, histidine, ornithine or mixtures thereof.

4. A method according to claim 3, in which the basic amino acid is lysine.

5. A method according to claim 1 in which the relaxer composition comprises at least one hydroxide ion generator which generates hydroxide ions in situ.
